(19) United States
(12) Patent Application Publication
(10) Pub. No.: US 2014/0057954 A1
(43) Pub. Date: Feb. 27, 2014

(54) CHEMICAL COMPOSITION

(71) Applicant: FOREST LABORATORIES HOLDINGS LTD., Hamilton (BM)

(72) Inventors: Anil Chhettry, Holtsville, NY (US); Mahendra G. Dedhiya, Pomona, NY (US); Kinjal Suchak, Perth Amboy, NJ (US); Stephan Ortiz, Lynbrook, NY (US); Chun Lin Chen, Brooklyn, NY (US)

(73) Assignee: FOREST LABORATORIES HOLDINGS LTD., Hamilton (BM)

(21) Appl. No.: 13/804,426

(22) Filed: Mar. 14, 2013

Related U.S. Application Data
(60) Provisional application No. 61/691,976, filed on Aug. 22, 2012.

(51) Int. Cl. A61K 31/41; A61K 31/353

(52) U.S. Cl. CPC A61K 31/41; A61K 31/353
USPC 514/381

(57) ABSTRACT

The present invention is directed to stable chemical compositions and dosage forms that comprise nebivolol and valsartan and which achieve therapeutically effective plasma levels of both actives in hypertensive patients following administration, as well as to methods of lowering blood pressure and treating hypertension using such compositions and dosage forms.

### FIELD OF THE INVENTION

The present invention is directed to stable chemical compositions and dosage forms that comprise nebivolol and valsartan and which achieve therapeutically effective plasma levels of both actives in hypertensive patients following administration, as well as to methods of lowering blood pressure and treating hypertension using such compositions and dosage forms.

### BACKGROUND OF THE INVENTION

High blood pressure, or hypertension, is a leading cause of heart attack, stroke, heart failure and kidney disease. In the U.S. alone, it is estimated that over 70 million adults have hypertension and that about 60% of hypertension sufferers have not achieved control of their symptoms. Accordingly, there is a strong and continual need for new therapies that better alleviate the symptoms of hypertension sufferers.

Applicants are developing a new therapy that combines valsartan and nebivolol (a unique beta receptor antagonist that additionally achieves vasodilation by stimulating nitric oxide release). This nebivolol/valsartan combination therapy is tremendously promising as it has been shown (see, e.g., in U.S. Pat. Nos. 7,803,838 and 7,838,552) to provide a synergistic effect in hypertensive rats when administered intravenously.

However, despite the high therapeutic potential of a drug product that combines nebivolol and valsartan, Applicants have encountered surprising and complex challenges in combining the two actives into a single therapeutically-effective oral dosage form.

For example, Applicants surprisingly discovered that when nebivolol is administered with valsartan even in free combination (as separate tablets), a pharmacokinetic (pK) interactive effect occurs which causes an unexpectedly large decrease in plasma levels of nebivolol. In addition, Applicants discovered that the pK interactive effect and decrease in nebivolol Cmax (maximum blood plasma concentration) may be further exacerbated by valsartan when the drugs are administered in a single dosage form. Moreover, as is appreciated in the art, nebivolol dissolution from dosage forms and thus nebivolol delivery may be further impacted by a variety of factors.

Overall, these issues rendered unpredictable (i) the determination of which plasma concentrations of nebivolol and valsartan achieve desired pharmacodynamic (pD)/therapeutic effects in patients and (ii) the development of a chemical composition or combination dosage form that achieves the desired plasma concentration of nebivolol and the desired therapeutic performance in patients. As is appreciated in the art, the lack of knowledge of the pK/pD relationship prohibits one having ordinary skill in the art from predicting which plasma concentrations will be therapeutically-effective with any reasonable expectation of success. See, e.g., In re Cyclobenzaprine, 676 F.3d 1063 (Fed. Cir. Apr. 16, 2012).

Accordingly, there is a strong need for oral pharmaceutical dosage forms comprising nebivolol and valsartan which achieve therapeutically-effective plasma concentrations of nebivolol and valsartan (despite the pK interactive effect) and which are well-suited for treating hypertension in patients in need thereof.

### SUMMARY OF THE INVENTION

Applicants have now developed stable oral pharmaceutical dosage forms of nebivolol and valsartan for achieving therapeutically-effective plasma concentrations of nebivolol and valsartan following administration to patients in need thereof.

In some embodiments, the application provides a method of treating hypertension in a patient, comprising administering to a patient diagnosed with hypertension an oral pharmaceutical dosage form that comprises a pharmaceutically acceptable carrier, about 5 mg to about 20 mg of nebivolol or a pharmaceutically acceptable salt thereof, about 80 mg to about 320 mg of valsartan or a pharmaceutically acceptable salt thereof, wherein the oral pharmaceutical dosage form upon administering to the patient provides a therapeutically effective plasma concentration of nebivolol for treating hypertension in the patient.

In some embodiments, the application provides a method of treating hypertension in a patient, comprising administering to a patient diagnosed with hypertension an oral pharmaceutical dosage form that comprises a pharmaceutically acceptable carrier, about 80 mg to about 320 mg of valsartan or a pharmaceutically acceptable salt thereof, and about 20 mg of nebivolol or a pharmaceutically acceptable salt thereof, wherein the oral pharmaceutical dosage form upon administering to the patient provides a nebivolol Cmax between about 3 ng/mL and about 5 ng/mL, an AUC_{0-∞} for nebivolol between about 35 ng·h/mL and about 60 ng·h/mL, and a Tmax for nebivolol between about 2 hours and about 4 hours, following oral administration of a single dose of the oral dosage form to a patient in need thereof.

In some embodiments, the application provides a method of treating hypertension in a patient, comprising administering to a patient diagnosed with hypertension an effective amount of an oral pharmaceutical dosage form that comprises a pharmaceutically acceptable carrier, about 80 mg to about 320 mg of valsartan or a pharmaceutically acceptable salt thereof, and about 20 mg of nebivolol or a pharmaceutically acceptable salt thereof, wherein the oral pharmaceutical dosage form upon administering to the patient provides a nebivolol Cmaxbetween about 3.5 ng/mL and about 4.3 ng/mL, an AUC_{0-∞} for nebivolol between about 40 ng·h/mL and about 50 ng·h/mL, and a Tmax for nebivolol between about 2.5 hours and about 3.5 hours, following oral administration of a single dose of the oral dosage form to a patient in need thereof.

In some embodiments, the application provides a method of treating hypertension in a patient, comprising administering to a patient diagnosed with hypertension an oral pharmaceutical dosage form that comprises a pharmaceutically acceptable carrier, about 80 mg to about 320 mg of valsartan or a pharmaceutically acceptable salt thereof, and about 10 mg of nebivolol or a pharmaceutically acceptable salt thereof, wherein the oral pharmaceutical dosage form upon administering to the patient provides a nebivolol Cmax between about 1.5 ng/mL and about 2.5 ng/mL, an AUC_{0-∞} for nebivolol between about 15 ng·h/mL and about 30 ng·h/mL, and a Tmax for nebivolol between about 2 hours and about 4 hours, following oral administration of a single dose of the oral dosage form to a patient in need thereof.

In some embodiments, the application provides a method of treating hypertension in a patient, comprising administering to a patient diagnosed with hypertension an oral pharmaceutical dosage form that comprises a pharmaceutically acceptable carrier, about 80 mg to about 320 mg of valsartan or a pharmaceutically acceptable salt thereof, and about 10 mg of nebivolol or a pharmaceutically acceptable salt thereof, wherein the oral pharmaceutical dosage form upon administering to the patient provides a nebivolol Cmax between about 1.8 ng/mL and about 2.2 ng/mL, an AUC_{0-∞} for nebivolol between about 20 ng·h/mL and about 25 ng·h/mL, and a Tmax for nebivolol between about 2.5 hours and about 3.5 hours, following oral administration of a single dose of the oral dosage form to a patient in need thereof.

In some embodiments, the application provides a method of treating hypertension in a patient, comprising administering to a patient diagnosed with hypertension an oral pharmaceutical dosage form that comprises a pharmaceutically acceptable carrier, about 80 mg to about 320 mg of valsartan or a pharmaceutically acceptable salt thereof, and about 5 mg of nebivolol or a pharmaceutically acceptable salt thereof, wherein the oral pharmaceutical dosage form upon administering to the patient provides a nebivolol Cmax between about 0.8 ng/mL and about 1.2 ng/mL, an AUC_{0-∞} for nebivolol between about 9.5 ng·h/mL and about 13 ng·h/mL, and a Tmax for nebivolol between about 2.3 hours and about 3.3 hours, following oral administration of a single dose of the oral dosage form to a patient in need thereof.

In some embodiments, the application provides a method of treating hypertension in a patient, comprising administering to a patient diagnosed with hypertension an oral pharmaceutical dosage form that comprises a pharmaceutically acceptable carrier, about 80 mg to about 320 mg of valsartan or a pharmaceutically acceptable salt thereof, and about 5 mg of nebivolol or a pharmaceutically acceptable salt thereof, wherein the oral pharmaceutical dosage form upon administering to the patient provides a nebivolol Cmax between about 0.9 ng/mL and about 1.1 ng/mL, an AUC_{0-∞} for nebivolol between about 10 ng·h/mL and about 12.3 ng·h/mL, and a Tmax for nebivolol between about 2.5 hours and about 3.1 hours, following oral administration of a single dose of the oral dosage form to a patient in need thereof.

In some embodiments, the application provides an oral pharmaceutical dosage form which comprises a pharmaceutically acceptable carrier, about 5 mg to about 20 mg of nebivolol or a pharmaceutically acceptable salt thereof, and about 80 mg to about 320 mg of valsartan or a pharmaceutically acceptable salt thereof, which (upon administration of a single dose of the dosage form to a patient in need thereof) provides a nebivolol Cmax of about 0.5 to about 6 ng/mL, an AUC_{0-∞} for nebivolol of about 5 to about 70 ng·h/mL, and a Tmax of 1 to about 5 hours.

In some embodiments, a method is provided for treating hypertension in a patient, comprising administering to a patient diagnosed with hypertension an oral pharmaceutical dosage form that comprises a pharmaceutically acceptable carrier, about 20 mg of nebivolol or a pharmaceutically acceptable salt thereof, and about 320 mg of valsartan or a pharmaceutically acceptable salt thereof, wherein the oral pharmaceutical dosage form upon administering to the patient provides a therapeutically effective plasma concentration of nebivolol for treating hypertension in the patient, and wherein the oral pharmaceutical dosage form provides a nebivolol Cmax between about 3 ng/mL and about 5 ng/mL following a single administration of the oral pharmaceutical dosage form to a patient in need thereof. In some of these embodiments, the oral pharmaceutical dosage form also provides a nebivolol AUC_{0-∞} for nebivolol between about 35 ng·h/mL and about 60 ng·h/mL, and a nebivolol Tmax between 2 to about 4 hours, following a single administration of the oral pharmaceutical dosage form to a patient in need thereof.

In some embodiments, a method is provided for treating hypertension in a patient, comprising administering to a patient diagnosed with hypertension an oral pharmaceutical dosage form that comprises a pharmaceutically acceptable carrier, about 10 mg of nebivolol or a pharmaceutically acceptable salt thereof, and about 160 mg or 320 mg of valsartan or a pharmaceutically acceptable salt thereof, wherein the oral pharmaceutical dosage form upon administering to the patient provides a therapeutically effective plasma concentration of nebivolol for treating hypertension in the patient, and wherein the oral pharmaceutical dosage form provides a nebivolol Cmax between about 1.5 ng/mL and about 2.5 ng/mL following a single administration of the oral pharmaceutical dosage form to a patient in need thereof. In some of these embodiments, the oral pharmaceutical dosage form also provides a nebivolol AUC_{0-∞} for nebivolol between about 15 ng·h/mL and about 30 ng·h/mL, and a nebivolol Tmax between 2 to about 4 hours, following a single administration of the oral pharmaceutical dosage form to a patient in need thereof.

In some embodiments, a method is provided for treating hypertension in a patient, comprising administering to a patient diagnosed with hypertension an oral pharmaceutical dosage form that comprises a pharmaceutically acceptable carrier, about 5 mg of nebivolol or a pharmaceutically acceptable salt thereof, and about 80 mg or 160 mg of valsartan or a pharmaceutically acceptable salt thereof, wherein the oral pharmaceutical dosage form upon administering to the patient provides a therapeutically effective plasma concentration of nebivolol for treating hypertension in the patient, and wherein the oral pharmaceutical dosage foini provides a nebivolol Cmax between about 0.75 ng/mL and about 1.25 ng/mL following a single administration of the oral pharmaceutical dosage form to a patient in need thereof. In some of these embodiments, the oral pharmaceutical dosage form also provides a nebivolol AUC_{0-∞} for nebivolol between about 7 ng·h/mL and about 15 ng·h/mL, and a nebivolol Tmax between 2 to about 4 hours, following a single administration of the oral pharmaceutical dosage form to a patient in need thereof.

In some preferred embodiments of these methods, the oral pharmaceutical dosage form releases at least 80% of the nebivolol contained therein after 30 minutes of entering a use environment.

In some embodiments, the application provides a chemical composition comprising a component having the structure of formula I:

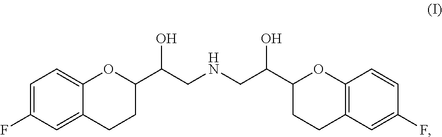 [SMILES: OC(CNCC(O)C1CCc2cc(F)ccc2O1)C1CCc2cc(F)ccc2O1]

a pharmaceutical carrier, and valsartan or a pharmaceutically acceptable salt thereof, wherein the chemical composition upon administering to a patient in need thereof provides a therapeutically effective plasma concentration of the compound of formula I.

### BRIEF DESCRIPTION OF THE FIGURES

FIG. 1 illustrates the comparative dissolution of nebivolol from a nebivolol monotherapy tablet and from the tablets prepared in Examples 1 and 2.

FIG. 2 illustrates the comparative nebivolol exposure achieved in subjects following administration of nebivolol and valsartan in free combination (separate valsartan and nebivolol tablets) versus in the oral combination tablet prepared in Example 1.

### DETAILED DESCRIPTION OF THE INVENTION

The present invention relates to stable oral dosage forms of valsartan and nebivolol achieve therapeutically-effective plasma concentrations of both valsartan and nebivolol following administration to hypertensive patients.

The terms “nebivolol” and “nebivolol hydrochloride” are used synonymously herein to refer to β1-selective β-blocker which is commercially available in monotherapy faun as Bystolic® (depicted in formula I) or any pharmaceutically acceptable salt thereof or free base thereof.

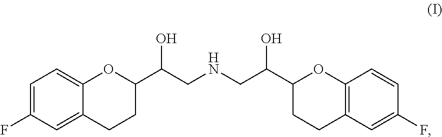 [SMILES: OC(CNCC(O)C1CCc2cc(F)ccc2O1)C1CCc2cc(F)ccc2O1]

The term “valsartan” refers to the nonpeptide and specific angiotensin II receptor antagonist which is commercially available in monotherapy form as Diovan® or any pharmaceutically acceptable salt thereof.

The term “pharmaceutically acceptable salt” is used herein to refer to any salt of valsartan or nebivolol that is physiologically tolerated by a patient. The term “entry into a use environment” is used herein, unless otherwise indicated, to refer to contact of the dosage form with gastric or intestinal fluid of a patient to whom it is administered, or with a fluid intended to simulate gastric or intestinal fluid (for example, with 900 mL of 0.1N hydrochloric acid solution at a temperature of 37° C. and subjected to agitation using USP apparatus II at 50 rpm, or about 900 mL of a buffered solution having a pH of about 6.8 and subjected to agitation using USP Apparatus II at about 50 rpm).

The terms “dissolution rate for nebivolol,” “dissolution rate for the nebivolol component,” “dissolution rate for valsartan,” “dissolution rate for the valsartan component” and variations thereof, are used herein unless otherwise indicated, to mean the dissolution rates of the nebivolol and valsartan components of the dosage form, respectively, out of the dosage form following the passage of a specified amount of time following entry into a use environment.

As used herein, unless otherwise indicated, dissolution rates define the percentage of valsartan or nebivolol originally contained in the dosage form that is released from the dosage form within a specified period of time following entry of the dosage form into a use environment.

The term “treating” is used herein, unless otherwise indicated, to mean to relieve, alleviate, delay, reduce, reverse, or improve at least one symptom of hypertension in a patient. The term “treating” may also mean to arrest, delay the onset (i.e., the period prior to clinical manifestation of a disease, disorder or condition) and/or reduce the risk of developing or worsening one or symptoms of hypertension.

The terms “effective amount” and “therapeutically effective amount” are used herein, unless otherwise indicated, to refer to an amount or quantity of nebivolol and/or valsartan which is sufficient to elicit an appreciable biological response when administered to a patient. For example, the terms “effective amount” and “therapeutically effective amount” refer to an amount of nebivolol and/or valsartan that, when administered to a patient (e.g., human or other mammal) for treating hypertension is sufficient to effect such treatment of one or more symptoms of hypertension. It will be appreciated that the precise therapeutic dose will depend on the age, condition, weight, use of concomitant drugs, etc. of the patient and will be ultimately be at the discretion of the attending physician.

Numerical ranges that are defined by the phrase “about [lower range value]-[upper range value]” should be understood, unless otherwise indicated, to define a numerical range “from about [the lower range value] to about [the upper range value],” inclusive of the lower and upper range values. Moreover, the term “about”, when used herein to refer to a specific value, should be understood to include values that are within 5% (in other words, +/−5%) of the specified value.

The term “bioequivalent” is used herein to mean that the 90% confidence intervals for the ratio of log transformed geometric means (e.g., AUC test/AUC reference) of the primary pK parameters is within the range of 80% to 125%.

The term “unit” is used herein to refer to a portion of the dosage form, for example, any region, microenvironment, layer, space, etc. of the dosage form or the like. For example, the dosage form in preferred embodiments comprises two or more distinct units, i.e., units that occupy separate/different space in the dosage form and comprise at least one different component. For example, in some preferred embodiments, the dosage form comprises at least one first unit that comprises nebivolol (or a salt thereof) and substantially no (or even no) valsartan and at least least one distinct second unit that comprises valsartan (or a salt thereof) and substantially no (or even no) nebivolol.

The term “combination dosage form” is used herein synonymously with the teinis fixed-dose combination dosage form, chemical composition and multi-unit dosage form.

The terms “a”; “the” and other similar singular designators, when used herein to refer to a specific component of the invention, should be understood as meaning that the invention may comprise one or more (e.g., two or more or even ten or more) of that component, unless otherwise indicated.

The phrase “consisting of” when used in reference to the dosage form or in reference to an individual unit of the dosage form, means that the oral dosage form or unit contains no other active pharmaceutical ingredients or excipients other than those specified but may contain additional components or steps that are unrelated to the invention and/or impurities ordinarily associated with the recited steps or components.

The phrase “consisting essentially of,” when used in reference to the dosage form or in reference to an individual unit of the dosage form, means that the dosage form or individual unit thereof contains no active pharmaceutical ingredients other than those specified, but that it may contain additional inactive components or excipients. For example, if a dosage form is described as consisting essentially of valsartan and nebivolol, it should be understood that the dosage form contains nebivolol, valsartan and no other active pharmaceutical ingredients, but that the dosage form may contain any number of additional inactive components or excipients. Moreover, if a dosage form is described as having a first unit that consists essentially of nebivolol, it should be understood that (i) the first unit includes no active pharmaceutical ingredients other than nebivolol but may contain any number of additional inactive components or excipients and (ii) the second unit of the dosage form may contain any number of active and inactive components (unless otherwise specified).

### DETAILED DESCRIPTION

This application provides oral pharmaceutical dosage forms comprising nebivolol and valsartan that are surprisingly suited for achieving therapeutically effective plasma levels of nebivolol and valsartan for treating hypertension in patients in need thereof (e.g., patients diagnosed with mild to moderate hypertension).

The inventive oral dosage form in some preferred embodiments provides a nebivolol Cmax (maximum blood plasma concentration) of about 0.25 to about 7 ng/mL, an AUC_{0-∞} for nebivolol of about 2 to about 80 ng·h/mL, and a Tmax of 1 to about 5 hours following oral administration of a single dose of the oral dosage form comprising valsartan and about 5 to about 20 mg of nebivolol.

The inventive oral dosage form in some preferred embodiments provides a nebivolol Cmax (maximum blood plasma concentration) of about 0.5 to about 6 ng/mL, an AUC_{0-∞} for nebivolol of about 5 to about 70 ng·h/mL, and a Tmax of 1 to about 5 hours following oral administration of a single dose of the oral dosage form comprising valsartan and about 5 to about 20 mg of nebivolol.

The inventive oral dosage form in some preferred embodiments provides a nebivolol Cmax (maximum blood plasma concentration) of about 0.75 to about 5 ng/mL, an AUC_{0-∞} for nebivolol of about 7 to about 60 ng·h/mL, and a Tmax of 1.5 to about 3.5 hours following oral administration of a single dose of the oral dosage form comprising valsartan and about 5 to about 20 mg of nebivolol.

The inventive oral dosage form in some preferred embodiments provides a nebivolol Cmax of about 2 to about 6 ng/mL following oral administration of a single dose of the oral dosage form comprising valsartan and about 20 mg of nebivolol. In some preferred embodiments, the oral dosage form provides a nebivolol Cmax (maximum blood plasma concentration) of about 3 to about 5 ng/mL following oral administration of a single dose of the oral dosage form comprising valsartan and about 20 mg of nebivolol. In some preferred embodiments, the oral dosage form provides a nebivolol Cmax (maximum blood plasma concentration) within the range of about 80% to about 125% of about 3.9 ng/mL following oral administration of a single dose of the oral dosage form comprising valsartan and about 20 mg of nebivolol.

The inventive oral dosage form in some preferred embodiments provides a nebivolol Cmax (maximum blood plasma concentration) of about 0.5 to about 4 ng/mL following oral administration of a single dose of the oral dosage form comprising valsartan and about 10 mg of nebivolol. In some preferred embodiments, the oral dosage form provides a nebivolol Cmax (maximum blood plasma concentration) of about 1 to about 3 ng/mL following oral administration of a single dose of the oral dosage form comprising valsartan and about 10 mg of nebivolol. In some preferred embodiments, the oral dosage form provides a nebivolol Cmax (maximum blood plasma concentration) of about 1.5 to about 2.5 ng/mL following oral administration of a single dose of the oral dosage form comprising valsartan and about 10 mg of nebivolol. In some preferred embodiments, the oral dosage form provides a nebivolol Cmax (maximum blood plasma concentration) within the range of about 80% to about 125% of about 2 ng/mL following oral administration of a single dose of the oral dosage form comprising valsartan and about 10 mg of nebivolol.

The inventive oral dosage form in some preferred embodiments provides a nebivolol Cmax (maximum blood plasma concentration) of about 0.1 to about 3 ng/mL following oral administration of a single dose of the oral dosage form comprising valsartan and about 5 mg of nebivolol. In some preferred embodiments, the oral dosage form provides a nebivolol Cmax (maximum blood plasma concentration) of about 0.5 to about 1.5 ng/mL following oral administration of a single dose of the oral dosage form comprising valsartan and about 5 mg of nebivolol. In some preferred embodiments, the oral dosage form provides a nebivolol Cmax (maximum blood plasma concentration) of about 0.75 to about 1.25 ng/mL following oral administration of a single dose of the oral dosage form comprising valsartan and about 5 mg of nebivolol. In some preferred embodiments, the oral dosage form provides a nebivolol Cmax (maximum blood plasma concentration) within the range of about 80% to about 125% of about 1 ng/mL following oral administration of a single dose of the oral dosage form comprising valsartan and about 5 mg of nebivolol.

The inventive oral dosage form in some preferred embodiments provides an AUC_{0-∞} for nebivolol of about 20 to about 70 ng·h/mL following oral administration of a single dose of the oral dosage form comprising valsartan and about 20 mg of nebivolol. In some preferred embodiments, the oral dosage form provides an AUC_{0-∞} for nebivolol of about 30 to about 65 ng·h/mL following oral administration of a single dose of the oral dosage form comprising valsartan and about 20 mg of nebivolol. In some preferred embodiments, the oral dosage form provides an AUC_{0-∞} for nebivolol of about 35 to about 60 ng·h/mL following oral administration of a single dose of the oral dosage foim comprising valsartan and about 20 mg of nebivolol. In some preferred embodiments, the oral dosage form provides an AUC_{0-∞} for nebivolol within the range of about 80% to about 125% of about 45 ng·h/mL following oral administration of a single dose of the oral dosage form comprising valsartan and about 20 mg of nebivolol.

The inventive oral dosage form in some preferred embodiments provides an AUC_{0-∞} for nebivolol of about 5 to about 40 ng·h/mL following oral administration of a single dose of the oral dosage form comprising valsartan and about 10 mg of nebivolol. In some preferred embodiments, the oral dosage form provides an AUC_{0-∞} for nebivolol of about 10 to about 35 ng·h/mL following oral administration of a single dose of the oral dosage form comprising valsartan and about 10 mg of nebivolol. In some preferred embodiments, the oral dosage form provides an AUC_{0-∞} for nebivolol of about 15 to about 30 ng·h/mL following oral administration of a single dose of the oral dosage form comprising valsartan and about 10 mg of nebivolol. In some preferred embodiments, the oral dosage form provides an AUC_{0-∞} for nebivolol within the range of about 80% to about 125% of about 22.5 ng·h/mL following oral administration of a single dose of the oral dosage form comprising valsartan and about 10 mg of nebivolol.

The inventive oral dosage form in some preferred embodiments provides an AUC_{0-∞} for nebivolol of about 5 to about 20 ng·h/mL following oral administration of a single dose of the oral dosage form comprising valsartan and about 5 mg of nebivolol. In some preferred embodiments, the oral dosage form provides an AUC_{0-∞} for nebivolol of about 7 to about 15 ng·h/mL following oral administration of a single dose of the oral dosage form comprising valsartan and about 5 mg of nebivolol. In some preferred embodiments, the oral dosage form provides an AUC_{0-∞} for nebivolol within the range of about 80% to about 125% of about 11.2 ng·h/mL following oral administration of a single dose of the oral dosage form comprising valsartan and about 5 mg of nebivolol.

The inventive oral dosage form in some preferred embodiments provides a Tmax for nebivolol of about 1 to about 5 hours following oral administration of a single dose of the oral dosage form comprising valsartan and about 20 mg of nebivolol. In some preferred embodiments, the oral dosage form provides a Tmax for nebivolol of about 1 to about 5 hours following oral administration of a single dose of the oral dosage form comprising valsartan and about 10 mg of nebivolol. In some preferred embodiments, the oral dosage form provides a Tmax for nebivolol of about 1 to about 5 hours following oral administration of a single dose of the oral dosage form comprising valsartan and about 5 mg of nebivolol.

In some preferred embodiments, the oral dosage form provides a Tmax for nebivolol of about 2 to about 4 hours following oral administration of a single dose of the oral dosage form comprising valsartan and about 20 mg of nebivolol.

In some preferred embodiments, the oral dosage form provides a Tmax for nebivolol of about 2 to about 4 hours following oral administration of a single dose of the oral dosage form comprising valsartan and about 10 mg of nebivolol.

In some preferred embodiments, the oral dosage form provides a Tmax for nebivolol of about 2 to about 4 hours following oral administration of a single dose of the oral dosage form comprising valsartan and about 5 mg of nebivolol.

In some preferred embodiments, the oral dosage form provides a nebivolol Cmax of about 3 to about 5 ng/mL, an AUC_{0-∞} for nebivolol of about 35 to about 60 ng·h/mL, and a Tmax for nebivolol of about 2 to about 4 hours following oral administration of a single dose of the oral dosage form comprising valsartan and about 20 mg of nebivolol. In some preferred embodiments, the oral dosage form provides a nebivolol Cmax of about 3.3 to about 4.9 ng/mL, an AUC_{0-∞} for nebivolol of about 38.2 to about 51.8 ng·h/mL, and a Tmax for nebivolol of about 2.3 to about 3.3 hours following oral administration of a single dose of the oral dosage form comprising valsartan and about 20 mg of nebivolol.

In some preferred embodiments, the oral dosage form provides a nebivolol Cmax between about 3.5 ng/mL and about 4.3 ng/mL, an AUC_{0-∞} for nebivolol between about 40 ng·h/mL and about 50 ng·h/mL, and a Tmax for nebivolol between about 2.5 hours and about 3.5 hours following oral administration of a single dose of the oral dosage form to a patient in need thereof.

In some preferred embodiments, the oral dosage form provides a nebivolol Cmax of about 3.5 to about 4.3 ng/mL, an AUC_{0-∞} for nebivolol of about 40.5 to about 49.5 ng·h/mL, and a Tmax for nebivolol of about 2.5 to about 3.1 hours following oral administration of a single dose of the oral dosage form comprising valsartan and about 20 mg of nebivolol.

In some preferred embodiments, the oral dosage form provides a nebivolol Cmax of about 3.7 to about 4.1 ng/mL, an AUC_{c}, for nebivolol of about 42.7 to about 47.3 ng·h/mL, and a Tmax for nebivolol of about 2.6 to about 3.0 hours following oral administration of a single dose of the oral dosage form comprising valsartan and about 20 mg of nebivolol.

In some preferred embodiments, the oral dosage form provides a nebivolol Cmax of about 80% to about 125% of about 3.9 ng/mL, an AUC_{0-∞} for nebivolol of about 80% to about 125% of about 45 ng·h/mL, and a Tmax for nebivolol of about 80% to about 125% of about 2.8 hours following oral administration of a single dose of the oral dosage form comprising valsartan and about 20 mg of nebivolol.

In some preferred embodiments, the oral dosage form provides a nebivolol Cmax of about 1.5 to about 2.5 ng/mL, an AUC_{0-∞} for nebivolol of about 15 to about 30 ng·h/mL, and a Tmax for nebivolol of about 2 to about 4 hours following oral administration of a single dose of the oral dosage form comprising valsartan and about 10 mg of nebivolol.

In some preferred embodiments, the oral dosage form provides a nebivolol Cmax of about 1.7 to about 2.3 ng/mL, an AUC_{0-∞} for nebivolol of about 19.1 to about 28.9 ng·h/mL, and a Tmax for nebivolol of about 2.3 to about 3.3 hours following oral administration of a single dose of the oral dosage form comprising valsartan and about 10 mg of nebivolol.

In some preferred embodiments, the oral dosage form provides a nebivolol Cmax between about 1.8 ng/mL and about 2.2 ng/mL, an AUC0-∞ for nebivolol between about 20 ng·h/mL and about 25 ng·h/mL, and a Tmax for nebivolol between about 2.5 hours and about 3.5 hours following oral administration of a single dose of the oral dosage form to a patient in need thereof.

In some preferred embodiments, the oral dosage form provides a nebivolol Cmax of about 1.8 to about 2.2 ng/mL, an AUC_{0-∞} for nebivolol of about 20.2 to about 24.8 ng·h/mL, and a Tmax for nebivolol of about 2.5 to about 3.1 hours following oral administration of a single dose of the oral dosage form comprising valsartan and about 10 mg of nebivolol.

In some preferred embodiments, the oral dosage form provides a nebivolol Cmax of about 1.9 to about 2.1 ng/mL, an AUC_{0-∞} for nebivolol of about 21.3 to about 23.7 ng·h/mL, and a Tmax for nebivolol of about 2.6 to about 3.0 hours following oral administration of a single dose of the oral dosage form comprising valsartan and about 10 mg of nebivolol.

In some preferred embodiments, the oral dosage form provides a nebivolol Cmax of about 80% to about 125% of about 2 ng/mL, an AUC_{0-∞} for nebivolol of about 80% to about 125% of about 22.5 ng·h/mL, and a Tmax for nebivolol of about 80% to about 125% of about 2.8 hours following oral administration of a single dose of the oral dosage form comprising valsartan and about 10 mg of nebivolol.

In some preferred embodiments, the oral dosage form provides a nebivolol Cmax of about 0.75 to about 1.25 ng/mL, an AUC_{0-∞} for nebivolol of about 7 to about 15 ng·h/mL, and a Tmax for nebivolol of about 2 to about 4 hours following oral administration of a single dose of the oral dosage form comprising valsartan and about 5 mg of nebivolol.

In some preferred embodiments, the oral dosage form provides a nebivolol Cmax of about 0.8 to about 1.2 ng/mL, an AUC_{0-∞} for nebivolol of about 9.5 to about 12.9 ng·h/mL, and a Tmax for nebivolol of about 2.3 to about 3.3 hours following oral administration of a single dose of the oral dosage form comprising valsartan and about 5 mg of nebivolol.

In some preferred embodiments, the oral dosage form provides a nebivolol Cmax of about 0.9 to about 1.1 ng/mL, an AUC_{0-∞} for nebivolol of about 10.0 to about 12.3 ng·h/mL, and a Tmax for nebivolol of about 2.5 to about 3.1 hours following oral administration of a single dose of the oral dosage form comprising valsartan and about 5 mg of nebivolol.

In some preferred embodiments, the oral dosage form provides a nebivolol Cmax of about 0.9 to about 1.1 ng/mL, an AUC_{0-∞} for nebivolol of about 10.6 to about 11.8 ng·h/mL, and a Tmax for nebivolol of about 2.6 to about 3.0 hours following oral administration of a single dose of the oral dosage form comprising valsartan and about 5 mg of nebivolol.

In some preferred embodiments, the oral dosage form provides a nebivolol Cmax of about 80% to about 125% of about 1 ng/mL, an AUC_{0-∞} for nebivolol of about 80% to about 125% of about 11.2 ng·h/mL, and a Tmax for nebivolol of about 80% to about 125% of about 2.8 hours following oral administration of a single dose of the oral dosage form comprising valsartan and about 5 mg of nebivolol.

In some embodiments, the oral dosage form achieves nebivolol exposure (or nebivolol plasma concentrations) in patients upon administration that is bioequivalent to the nebivolol exposure achieved by nebivolol and valsartan in free combination (separately administered tablets) at the same dosage.

### Dissolution Rate of the Dosage Forms

The dosage forms have been found to provide desirable dissolution rates for valsartan and nebivolol following entry into a use environment. In some preferred embodiments, the dosage form provides a dissolution rate for nebivolol of at least about 70% after 30 minutes following entry into a use environment. In some embodiments, the dosage form provides a dissolution rate for nebivolol of at least about 75% after 30 minutes following entry into a use environment. In some embodiments, the dosage form provides a dissolution rate for nebivolol of at least about 80% after 30 minutes following entry into a use environment.

In some preferred embodiments, the dosage form provides a dissolution rate for nebivolol of at least about 60% after 15 minutes following entry into a use environment. In some preferred embodiments, the dosage form provides a dissolution rate for nebivolol of at least about 70% after 15 minutes following entry into a use environment. In some embodiments, the dosage form provides a dissolution rate for nebivolol of at least about 75% after 15 minutes following entry into a use environment. In some embodiments, the dosage form provides a dissolution rate for nebivolol of at least about 80% after 15 minutes following entry into a use environment.

In some preferred embodiments, the dosage form provides a dissolution rate for nebivolol of at least about 70% after 30 minutes following entry into about 900 mL of 0.1N HCl solution at a temperature of about 37° C. and subjected to agitation using USP Apparatus II at about 50 rpm. In some embodiments, the dosage form provides a dissolution rate for nebivolol of at least about 75% after 30 minutes following entry into about 900 mL of 0.1N HCl solution at a temperature of about 37° C. and subjected to agitation using USP Apparatus II at about 50 rpm. In some embodiments, the dosage form provides a dissolution rate for nebivolol of at least about 80% after 30 minutes following entry into about 900 mL of 0.1N HCl solution at a temperature of about 37° C. and subjected to agitation using USP Apparatus II at about 50 rpm.

In some preferred embodiments, the dosage form provides a dissolution rate for nebivolol of at least about 70% after 15 minutes following entry into about 900 mL of 0.1N HCl solution at a temperature of about 37° C. and subjected to agitation using USP Apparatus II at about 50 rpm. In some embodiments, the dosage form provides a dissolution rate for nebivolol of at least about 75% after 15 minutes following entry into about 900 mL of 0.1N HCl solution at a temperature of about 37° C. and subjected to agitation using USP Apparatus II at about 50 rpm. In some embodiments, the dosage form provides a dissolution rate for nebivolol of at least about 80% after 15 minutes following entry into about 900 mL of 0.1N HCl solution at a temperature of about 37° C. and subjected to agitation using USP Apparatus II at about 50 rpm.

In some preferred embodiments, the dosage form provides a dissolution rate for valsartan between about 0.01% and about 10% after 15 minutes (for example, after 30 minutes, after 45 minutes or even after 60 minutes) following entry into about 900 mL of a buffered solution having a pH of about 6.8 and a temperature of about 37° C. and subjected to agitation using USP Apparatus II at about 50 rpm. In some preferred embodiments, the dosage form provides a dissolution rate for valsartan between about 0.1% and about 5% after 15 minutes (for example, after 30 minutes, after 45 minutes or even after 60 minutes) following entry into about 900 mL of a buffered solution having a pH of about 6.8 and a temperature of about 37° C. and subjected to agitation using USP Apparatus II at about 50 rpm. In some preferred embodiments, the dosage form provides a dissolution rate for valsartan between about 0.1% and about 3% after 15 minutes following entry into about 900 mL of a buffered solution having a pH of about 6.8 and a temperature of about 37° C. and subjected to agitation using USP Apparatus II at about 50 rpm.

### Dosage and Excipients

The dosage form comprises a first unit (e.g., a first region, microenvironment, layer, portion, space, etc., or the like) that comprises nebivolol (or a salt thereof); and a second unit (e.g., a second distinct unit, region, microenvironment, layer, portion, space, etc., or the like) that comprises valsartan (or a salt thereof). In some preferred embodiments, the dosage form comprises a first unit that comprises nebivolol and substantially no (or even no) valsartan; and a second unit that comprises valsartan and substantially no (or even no) nebivolol. As would be appreciated, there are several different ways to configure such a multi-unit dosage form, such as for example in the form of a multi-layer dosage form, a bi-layer dosage form, a coated core dosage form, a multi-particulate oral dosage form, or similar suitable like configurations which are suitable for oral administration.

In some preferred embodiments, the dosage form is an oral pharmaceutical bi-layer dosage form.

The dosage form can comprise any suitable amount of nebivolol. In preferred embodiments, the dosage form comprises from about 0.1 mg to about 80 mg of nebivolol. In some embodiments, the dosage form comprises from about 0.5 mg to about 40 mg of nebivolol. Preferably, the dosage form comprises from about 2.5 mg to about 20 mg of nebivolol such as, for example, 2.5 mg+/−5% of nebivolol, 5 mg+/−5% of nebivolol, 10 mg+/−5% of nebivolol, or 20 mg+/−5% of nebivolol.

Moreover, the dosage form can comprise any suitable amount of valsartan. In some preferred embodiments, the dosage form comprises from about 10 mg to about 400 mg of valsartan. Preferably, the dosage form comprises from about 40 mg to about 320 mg of valsartan such as, for example, 40 mg+/−5% of valsartan, 80 mg+/−5% of valsartan, 160 mg+/−5% of valsartan, or 320 mg+/−5% of valsartan of valsartan.

In some preferred embodiments, the dosage form comprises about 5 mg of nebivolol and about 40 mg of valsartan. In some preferred embodiments, the dosage form comprises about 5 mg of nebivolol and about 80 mg of valsartan. In some preferred embodiments, the dosage form comprises about 5 mg of nebivolol and about 160 mg of valsartan. In some preferred embodiments, the dosage form comprises about 5 mg of nebivolol and about 320 mg of valsartan. In some preferred embodiments, the dosage form comprises about 10 mg of nebivolol and about 80 mg of valsartan. In some preferred embodiments, the dosage form comprises about 10 mg of nebivolol and about 160 mg of valsartan. In some preferred embodiments, the dosage form comprises about 10 mg of nebivolol and about 320 mg of valsartan. In some preferred embodiments, the dosage form comprises about 20 mg of nebivolol and about 80 mg of valsartan. In some preferred embodiments, the dosage form comprises about 20 mg of nebivolol and about 160 mg of valsartan. In some preferred embodiments, the dosage form comprises 20 mg of nebivolol and 320 mg of valsartan.

The dosage forms also comprise a suitable pharmaceutically acceptable carrier, meaning any one or more excipients or additives needed to achieve the desired stability, dissolution and pK/pD performance of the dosage form. The pharmaceutically acceptable carrier may include, without limitation, suitable diluents or fillers, disintegrants (e.g., superdisintegrants), glidants or lubricants, binders, surfactants, colorants, film coatings, and combinations or mixtures thereof. In some embodiments, the dosage form comprises nebivolol, valsartan, and a binder. In some embodiments, the dosage form comprises nebivolol, valsartan, and a superdisintegrant. In some embodiments, the dosage form comprises nebivolol, valsartan, a binder and a disintegrant.

Suitable disintegrants or superdisintegrants for use in the dosage form (e.g., one or more units of the dosage form) include, without limitation, sodium starch glycolate, crospovidone, croscarmellose (e.g., croscarmellose sodium), L-hydroxy propyl cellulose, and the like, and combinations or mixtures thereof.

When present, the disintegrant(s) or superdisintegrant(s) may be included in individual units of the dosage form in an amount ranging from about 0.1% by weight to about 20% by weight. In some embodiments, the concentration of the one or more disintegrant(s) (or superdisintegrants) in individual units of the dosage form ranges from about 0.5% by weight to about 15% by weight. In some embodiments, the concentration of the one or more disintegrants(s) (or superdisintegrants) in individual units is from about 0.5% by weight to about 10% by weight. In some embodiments, the concentration of the disintegrant(s) (or superdisintegrants) included in individual units is from about 1% by weight to about 5% by weight. In some embodiments, the concentration of the disintegrant(s) (or superdisintegrants) included in individual units is from about 5% by weight to about 10% by weight, for example from about 6% by weight to about 9% by weight. In some embodiments, the dosage form comprises a first unit than comprises valsartan and about 5% by weight to about 10% by weight of a disintegrant or superdisintegrant; and a second unit than comprises nebivolol and about 1% by weight to about 5% by weight of the same or a different disintegrant or superdisintegrant.

In some embodiments, the dosage form comprises a first unit that comprises valsartan and a disintegrant (or superdisintegrant) selected from sodium starch glycolate, crospovidone, croscarmellose sodium, and combinations thereof; and a second unit that comprises nebivolol and a disintegrant (or superdisintegrant) selected from sodium starch glycolate, crospovidone, croscarmellose sodium, and combinations thereof.

In some preferred embodiments, the dosage form is a multi-unit dosage form, wherein the first and second units both comprise a crospovidone or croscarmellose (e.g., croscamellose sodium) binder. In some embodiments, the dosage form comprises a first unit comprising valsartan and a second unit comprising nebivolol, wherein the first and second units both comprise crospovidone. In some embodiments, the dosage form comprises a first unit comprising valsartan and a second unit comprising nebivolol, wherein the first and second units both comprise croscarmellose (e.g., croscarmellose sodium).

Suitable binders for use in one or more units of the dosage form include, without limitation, polyvinylpyrrolidone, hydroxypropylmethyl cellulose (e.g., Methocel E5 or Methocel E15), hydroxypropyl cellulose, polyvinylpyrridone based binders (e.g., copovidone or crospovidone), pregelatinized starch, and the like, and combinations or mixtures thereof. In some embodiments, the dosage form comprises a first unit comprising valsartan and hydroxypropyl methylcellulose and a second unit that comprises nebivolol and copovidone.

When present, the binder(s) may be included in individual units of the dosage form in an amount ranging from about 0.1% by weight to about 10% by weight. In some embodiments, the concentration of the binder(s) included in individual units of the dosage form ranges from about 0.5% by weight to about 8% by weight. In some embodiments, the concentration of the binder(s) included in individual units of the dosage form ranges from about 1% by weight to about 5% by weight. In some embodiments, the dosage form comprises a first unit than comprises valsartan and about 0.5% by weight to about 5% by weight (e.g., about 1% by weight to about 4% by weight or even about 1% by weight to about 3% by weight) of a binder; and a second unit than comprises nebivolol and about 1% by weight to about 8% by weight (e.g., about 2% by weight to about 6% by weight or even about 3% by weight to about 5% by weight) of the same or a different binder.

Suitable fillers or diluents for use in one or more units of the dosage form include, without limitation, microcrystalline cellulose, lactose monohydrate, starch 1500, mannitol, sucrose or other sugars or sugar derivatives, low-substituted hydroxypropyl cellulose, and the like, and combinations or mixtures thereof. In some embodiments, the dosage form comprises a first unit than comprises valsartan and a diluent or filler that is selected from the group consisting of microcrystalline cellulose, lactose monohydrate, starch and combinations thereof. In some embodiments, the dosage form comprises a nebivolol unit than comprises nebivolol and a diluent or filler that is selected from the group consisting of microcrystalline cellulose, lactose monohydrate, starch and combinations thereof. In some preferred embodiments, the dosage foiin comprises a first unit that comprises valsartan and microcrystalline cellulose and a second unit that comprises nebivolol and lactose monohydrate.

When present, the filler or diluent may be included in individual units of the dosage form in any suitable amount. In some embodiments, the concentration of filler or diluent included in individual units of the dosage form ranges from about 0.5% by weight to about 40% by weight. In some embodiments, the concentration of filler or diluent included in individual units of the dosage form ranges from about 5% by weight to about 35% by weight. In some embodiments, the concentration of filler or diluent included in individual units of the dosage form ranges from about 10% by weight to about 30% by weight.

In some embodiments, the dosage form comprises a first unit than comprises valsartan and between about 5% by weight and about 25% by weight of a filler or diluent; and a second unit than comprises nebivolol and between about 15% by weight and about 40% by weight of the same or a different filler or diluent.

In some embodiments, the dosage form comprises a first unit than comprises valsartan and between about 10% by weight and about 18% by weigh of a filler or diluent; and a second unit than comprises nebivolol and between about 20% by weight and about 35% by weigh of the same or a different filler or diluent.

In some embodiments, the dosage form comprises a first unit than comprises valsartan and between about 12% by weight and about 16% by weight of a filler or diluent; and a second unit than comprises nebivolol and between about 25% by weight and about 30% by weight of the same or a different filler or diluent.

Suitable glidants for use in one or more units of the dosage form include, without limitation, colloidal silicon dioxide, magnesium trisilicate, powdered cellulose, starch, talc, and the like, and combinations or mixtures thereof. When present, one or more glidants may be included in individual units of the dosage form in any suitable amount. In some embodiments, the total concentration of glidant(s) in individual units of the dosage form ranges from about 0.1% by weight to about 4% by weight. In some embodiments, the total concentration of glidant(s) in individual units of the dosage form ranges from about 0.5% by weight to about 3% by weight. In some embodiments, the total concentration of glidant(s) in individual units of the dosage form ranges from about 1% by weight to about 3% by weight.

Suitable lubricants for use in one or more units of the dosage form include, without limitation, magnesium lubricants (e.g., magnesium stearate), aluminum lubricants (e.g., aluminum silicate), calcium lubricants (e.g., calcium silicate), stearic acid, cutina, PEG 4000-8000, talc, and the like, and combinations or mixtures thereof. When present, one or more lubricants may be included in individual units of the dosage form in any suitable amount. In some embodiments, the total concentration of lubricant(s) in individual units of the dosage form ranges from about 0.1% by weight to about 4% by weight. In some embodiments, the total concentration of lubricant(s) in individual units of the dosage form ranges from about 0.5% by weight to about 3% by weight. In some embodiments, the total concentration of lubricant(s) in individual units of the dosage form ranges from about 1% by weight to about 3% by weight.

Suitable colorants for use in one or more units of the dosage form include, without limitation, iron oxides such as blue, yellow, white, red, and black iron oxide, and the like, and combinations or mixtures thereof. When present, a colorant may be employed in an amount ranging from about 0.01% to about 1% by weight (e.g., from about 0.05% to about 0.15% by weight) of the solid dosage form (prior to any optional film coating).

Suitable film coatings for use in one or more units of the dosage form are known and commercially available or can be made according to known methods. Typically the film coating material is a polymeric film coating material comprising materials such as hydroxypropylmethyl cellulose, polyethylene glycol, talc and colorant. Typically, a film coating material is applied in such an amount as to provide a film coating that ranges from about 1% to about 6% by weight (e.g., from about 2% to about 4% by weight) of the film-coated tablet.

In some preferred embodiments, the dosage form comprises a first unit which comprises valsartan and a second unit which comprises nebivolol, wherein the first and second units each comprise a filler, a disintegrant (or superdisintegrant) and a binder.

In some preferred embodiments, the dosage form comprises a first unit which comprises valsartan, a surfactant and a disintegrant or superdisintegrant; and a second unit which comprises nebivolol, a binder and a lubricant. In some preferred embodiments, the dosage form comprises a first unit which comprises valsartan, a filler, a surfactant, a disintegrant (or superdisintegrant), a binder, and a lubricant; and a second unit which comprises nebivolol, a filler, a disintegrant (or superdisintegrant), a binder, and a lubricant.

In some preferred embodiments, the dosage form comprises a first unit which comprises between about 65% by weight and 85% by weight (e.g., between about 70% by weight and 80% by weight) of valsartan, and between about 3% by weight and about 12% by weight (e.g., between about 5% by weight and 10% by weight) of a disintegrant or superdisintegrant; and a second unit which comprises between about 0.5% by weight and about 7.5% by weight (e.g., between about 2% by weight and 6% by weight) of nebivolol, and between about 0.5% by weight and about 10% by weight (e.g., between about 1% by weight and 5% by weight) of a disintegrant (or superdisintegrant).

In some preferred embodiments, the dosage form comprises a first unit which comprises between about 65% by weight and 85% by weight (e.g., between about 70% by weight and 80% by weight) of valsartan, between about 3% by weight and about 12% by weight (e.g., between about 5% by weight and 10% by weight) of a disintegrant or superdisintegrant, and between about 0.5% by weight and about 5% by weight (e.g., between about 1% by weight and 3% by weight) of a binder; and a second unit which comprises between about 0.5% by weight and about 7.5% by weight (e.g., between about 2% by weight and 6% by weight) of nebivolol, between about 1% by weight and 10% by weight (e.g., between about 2% by weight and 6% by weight) of a binder; and between about 0.5% by weight and about 10% by weight (e.g., between about 1% by weight and 5% by weight) of a disintegrant (or superdisintegrant).

In some preferred embodiments, the dosage form comprises a first unit which comprises between about 65% by weight and 85% by weight of valsartan, between about 3% by weight and about 12% by weight of a disintegrant (or superdisintegrant), between about 0.5% by weight and about 5% by weight of a binder; between about 10% by weight and 20% by weight of a filler, between about 0.01% by weight and about 2% by weight of a surfactant; and a second unit which comprises between about 0.5% by weight and about 7.5% by weight of nebivolol, between about 10% by weight and about 90% by weight of a filler, between about 1% by weight and 10% by weight of a binder; and between about 0.5% by weight and about 10% by weight of a disintegrant (or superdisintegrant).

In some preferred embodiments, the dosage form comprises a first unit which comprises between about 70% by weight and 80% by weight of valsartan, between about 5% by weight and 10% by weight of a disintegrant (or superdisintegrant), between about 1% by weight and 3% by weight of a binder; between about 10% by weight and 20% by weight of a filler, between about 0.1% by weight and 1% by weight of a surfactant; and a second unit which comprises between about 2% by weight and 6% by weight of nebivolol, between about 10% by weight and 35% by weight of a filler, between about 40% by weight and 80% by weight of a filler/binder/disintegrant agent (e.g., microcrystalline cellulose)), between about 2% by weight and 6% by weight of a binder; and between about 1% by weight and 5% by weight of a disintegrant or superdisintegrant.

The dosage form can be prepared in any suitable configuration to achieve the desired pK performance. For example, in some preferred embodiments, the oral dosage form comprises two or more distinct units each containing at least one different active or inactive component. In some embodiments, the dosage form is a bi-layer dosage form (e.g., a bi-layer tablet) that comprises a first layer comprising nebivolol and a second layer comprising valsartan. In some embodiments, the dosage form is a coated dosage form (e.g., a coated tablet) that comprises a core region comprising valsartan and an outer coating than comprises nebivolol.

In some preferred embodiments, the dosage form comprises a valsartan tablet that is coated with a nebivolol-containing layer or unit. In some preferred embodiments, the dosage form comprises valsartan beads that are coated with a nebivolol-containing layer or unit. In some preferred embodiments, the dosage form comprises a valsartan composition (such as a valsartan tablet or valsartan beads) that comprise embedded nebivolol particles. In some preferred embodiments, the dosage form comprises a valsartan tablet (or a valsartan bead) that contains a core that comprises a nebivolol composition. In some preferred embodiments, the dosage form comprises a nebivolol tablet (or a nebivolol bead) that contains a core that comprises a valsartan composition.

In some preferred embodiments, the dosage form is a capsule that comprises (i) beads or granules that comprise (or consist of or consist essentially of) nebivolol or a salt thereof and one or more pharmaceutically acceptable carriers or excipients and (ii) distinct beads or granules that comprise (or consist of or consist essentially of) valsartan or a salt thereof and one or more pharmaceutically acceptable carriers or excipients. In some embodiments, the oral dosage form comprises a unit layer that includes nebivolol and substantially no (or no) valsartan. Alternatively, or in addition in some embodiments, the oral dosage form comprises a unit that includes valsartan and substantially no (or no) nebivolol.

### Methods Involving the Dosage Forms

The present invention also provides methods for treating hypertension (e.g., one or more symptoms of hypertension) in a patient diagnosed with hypertension. Moreover, present invention also provides methods for lowering blood pressure in a patient diagnosed with high blood pressure. These methods comprise, inter alia, diagnosing the patient with hypertension or high blood pressure and administering a therapeutically effective amount of the dosage form to a patient who has been diagnosed with the condition or disorder.

The methods may further comprise monitoring the patient for therapeutic efficacy and/or the occurrence of adverse events (for example, headaches, fatigue, dizziness, viral infection, and/or abdominal pain) following administration of a first dosage of the dosage form (e.g., repeated administrations over a prescribed period of treatment of the first dosage); and optionally administering a second dosage of the dosage form which is higher or lower than the first dosage. In some particularly preferred embodiments, the administered dosage is changed (increased or decreased) at two-week intervals. In some particular preferred embodiments, the monitoring step is performed at two-week intervals (for example, two weeks after administration of the initial dose).

For example, the treatment method may comprise administering a dosage form comprising a first dosage of valsartan and nebivolol for an first treatment period (e.g., once-daily for a two-day period, four-day period, one-week period or two-week period), monitoring the patient for therapeutic efficacy and/or the occurrence of adverse events during and/or following this period, and optionally administering a second dosage form having an increased or decreased dosage of valsartan and/or nebivolol. In some particularly preferred embodiments, the administered dosage is changed (increased or decreased) at two-week intervals. In some particular preferred embodiments, the monitoring step is performed at two-week intervals (for example, two weeks after administration of the initial dose).

In some embodiments, the treatment method may comprise administering a dosage form comprising about 5 mg of nebivolol and about 80 mg of valsartan for an first treatment period (e.g., once-daily for a two-day period, four-day period, one-week period or two-week period), monitoring the patient for therapeutic efficacy and/or the occurrence of adverse events during and/or following this period, and optionally administering a second dosage form comprising about 5 mg or 10 mg of nebivolol and about 160 mg of valsartan. In some particularly preferred embodiments, the administered dosage is changed (increased or decreased) at two-week intervals. In some particular preferred embodiments, the monitoring step is performed at two-week intervals (for example, two weeks after administration of the initial dose).

For example, the treatment method may comprise administering a dosage form comprising about 5 mg or 10 mg of nebivolol and about 160 mg of valsartan for an first treatment period (e.g., once-daily for a two-day period, four-day period, one-week period or two-week period), monitoring the patient for therapeutic efficacy and/or the occurrence of adverse events during and/or following this period, and optionally administering a second dosage form comprising: 10 mg of nebivolol and 160 mg of valsartan; 10 mg of nebivolol and 320 mg of valsartan; or 20 mg of nebivolol and 320 mg of valsartan. In some particularly preferred embodiments, the administered dosage is changed (increased or decreased) at two-week intervals. In some particular preferred embodiments, the monitoring step is performed at two-week intervals (for example, two weeks after administration of the initial dose).

In some preferred embodiments, extra care should be exercised when choosing the initial dose and dosing regimen for patients diagnosed with a biliary obstructive disorder, with mild or severe renal impairment, and/or with mild or moderate liver insufficiency. In this regard, the present invention comprises a method of treating hypertension in a patient diagnosed with a biliary obstructive disorder, with mild or severe renal impairment, and/or with mild or moderate liver/renal insufficiency (or impairment), wherein the method comprises administering a dosage form that comprises, for example, about 5 mg of nebivolol and about 80 mg of valsartan for an first treatment period (e.g., once-daily for a two-day period, four-day period, one-week period or two-week period), monitoring the patient for therapeutic efficacy and/or the occurrence of adverse events during and/or following this period, and optionally administering a second dosage form comprising about 5 mg or 10 mg of nebivolol and about 160 mg of valsartan. In some particularly preferred embodiments, the administered dosage is changed (increased or decreased) at two-week intervals. In some particular preferred embodiments, the monitoring step is performed at two-week intervals (for example, two weeks after administration of the initial dose). In some preferred embodiments, the method comprises administering a low or reduced dosage of the dosage form, monitoring the patient for therapeutic efficacy and/or the occurrence of adverse events during and/or following an initial treatment period, and optionally administering an increased dosage of the dosage form.

In this regard, the present invention comprises a method of treating hypertension in a patient diagnosed with diabetes, wherein the method comprises administering the dosage form, monitoring glucose levels in the patient following administration, and optionally adjusting the dosage of the dosage form for future administrations. In some particularly preferred embodiments, the administered dosage is changed (increased or decreased) at two-week intervals. In some particular preferred embodiments, the monitoring step is performed at two-week intervals (for example, two weeks after administration of the initial dose).

In this regard, the present invention comprises a method of treating hypertension in a patient diagnosed with pheochromocytoma, wherein the method comprises initiating treatment an alpha blocker prior to administering the dosage form.

In some preferred embodiments, a method is provided for treating hypertension in patients who are being coadministered a CYP2D6 inhibitor (e.g., quinidine, propafenone, fluoxetine, paroxetine, or the like), an inhibitor of hepatic uptake transporter OATP1B1 (e.g., rifampin or cyclosporine), an inhibitor of hepatic efflux transporter MRP2 (such as ritonavir), a catecholamine-depleting drug (e.g., reserpine or guanethidine), a digitalis glycoside, another beta blocker, an inhibitors of CYP 450 isoenzyme(s), a calcium channel blocker (e.g., a non-dihydropyridine calcium channel blocker such as verapamil or diltiazem), a myocardial depressant or inhibitor of AV conduction (such as certain calcium antagonists, particularly of the phenylalkylamine or benzothiazepine classes such as verapamil or diltiazem), an antiarrhythmic agent (e.g., disopyramide), a drug that block angiotensin II or its effects, a potassium sparing diuretic (e.g., spironolactone, triamterene, amiloride), a potassium supplement, or a salt substitute containing potassium, wherein the method comprises administering a first dosage of the dosage form (e.g., an initial low dose of the dosage form such as a dosage form comprising 5 mg of nebivolol and 80 mg of valsartan) for an first treatment period (e.g., once-daily for a two-day period, four-day period, one-week period or two-week period), monitoring the patient for therapeutic efficacy and/or the occurrence of adverse events during and/or following this period, and optionally administering a second dosage form having an increased or decreased dosage of valsartan and/or nebivolol. In some particularly preferred embodiments, the administered dosage is changed (increased or decreased) at two-week intervals. In some particular preferred embodiments, the monitoring step is performed at two-week intervals (for example, two weeks after administration of the initial dose). In some preferred embodiments, the method comprises administering a low or reduced dosage of the dosage form, monitoring the patient for therapeutic efficacy and/or the occurrence of adverse events during and/or following an initial treatment period, and optionally administering an increased dosage of the dosage form.

In some embodiments, the hypertension treatment method may achieve a lower incidence or rate of headaches as compared with nebivolol monotherapy having the same dosage. For example, in some embodiments the method may achieve at least about a 5% (preferably at least about 10%, at least about 20% or even at least about 30%) fewer (e.g., lower incidence of) headaches as compared with a nebivolol monotherapy of the same dosage (e.g., as measured across any suitable sample size of patients).

In some embodiments, the hypertension treatment method may achieve a lower incidence or rate of fatigue as compared with nebivolol monotherapy having the same dosage. For example, in some embodiments the method may achieve at least about a 5% (preferably at least about 10%, at least about 20% or even at least about 30%) less (e.g., lower incidence of) fatigue as compared with a nebivolol monotherapy of the same dosage (e.g., as measured across any suitable sample size of patients).

In some embodiments, the hypertension treatment method may achieve a lower incidence or rate of viral infection as compared with nebivolol monotherapy having the same dosage. For example, in some embodiments the method may achieve at least about a 5% (preferably at least about 10%, at least about 20% or even at least about 30%) lower incidence of viral infection as compared with a nebivolol monotherapy of the same dosage (e.g., as measured across any suitable sample size of patients).

In some embodiments, the hypertension treatment method may achieve a lower incidence or rate of abdominal pain as compared with nebivolol monotherapy having the same dosage. For example, in some embodiments the method may achieve at least about a 5% (preferably at least about 10%, at least about 20% or even at least about 30%) lower incidence of abdominal pain as compared with a nebivolol monotherapy of the same dosage (e.g., as measured across any suitable sample size of patients).

In some embodiments, the hypertension treatment method may achieve a lower incidence or rate of dizziness as compared with nebivolol monotherapy having the same dosage. For example, in some embodiments the method may achieve at least about a 5% (preferably at least about 10%, at least about 20% or even at least about 30%) less dizziness as compared with a nebivolol monotherapy of the same dosage (e.g., as measured across any suitable sample size of patients).

The method of treatment may comprise monitoring the patient for excessive hypotension and, if it occurs and/or is diagnosed, then reducing the dosage of the dosage form for future administrations.

The present invention also provides methods for preparing the dosage form. For example, valsartan can be prepared by any suitable method such as, but not limited to, dry compression or wet granulation or the like.

For example, the method may comprise mixing valsartan, a disintegrant or superdisintegrant and a filler (such as in a high shear granulator) to form a premix; granulating the premix by contacting the premix with a granulating solution that comprises a binder and a surfactant to form wetted granules; screening the wetted granules; drying the screened wetted granules to form dried granules; milling the dried granules; and then mixing (e.g., blending) the milled granules with a glidant, a lubricant and optionally a disintegrant or superdisintegrant.

In some embodiments, nebivolol is mixed or blended (e.g., sequentially blended) with a filler, a binder, a disintegrant or superdisintegrant, a glidant, and a lubricant.

In those embodiments in which valsartan and nebivolol compositions are at first prepared separately, these compositions may be subsequently combined into a single dosage form in any suitable manner to achieve any desired physical properties (e.g. hardness and/or appearance).

The following examples are merely illustrative of the present invention and should not be construed as limiting the scope of the invention in any way as many variations and equivalents that are encompassed by the present invention will become apparent to those skilled in the art upon reading the present disclosure.

### EXAMPLES

### Example 1

### Preparation of an Oral Pharmaceutical Multi-Unit Dosage Form of Valsartan and Nebivolol

Valsartan was mixed in a high shear granulator with microcrystalline cellulose and croscarmellose sodium to form a premix. The premix was granulated using a solution that contained hypromellose and polysorbate 80. The wetted granules were screened using a Frewitt mill to deagglomerate the granules and then dried in a Glatt fluid bed dryer to final LOD of about 2% to about 3%. The dried granules were milled using a Fitzmill or Comill and then mixed in a blender with croscarmellose sodium, magnesium stearate, talc, and silicon dioxide to produce a final blend.

Separately, nebivolol hydrochloride was mixed in a direct blending process (using a V-blender) with lactose monohydrate and silicified microcrystalline cellulose and then with polyvinylpyrrolidone, silicified microcrystalline cellulose, croscarmellose sodium, silicon dioxide, magnesium stearate, and a colorant.

The valsartan and nebivolol blends were then compressed (in this case, using a bilayer press) into the following multiple strength tablets: a tablet comprising about 5 mg nebivolol hydrochloride and about 80 mg of valsartan; a tablet having about 5 mg nebivolol hydrochloride and about 160 mg of valsartan; a tablet having about 10 mg nebivolol hydrochloride and about 160 mg of valsartan; a bi-layer tablet having about 5 mg nebivolol and about 320 mg of valsartan; a tablet having about 10 mg nebivolol hydrochloride and about 320 mg of valsartan; and a tablet having about 20 mg nebivolol hydrochloride and about 320 mg of valsartan. Depending on the dosage strength, these dosage forms had a hardness of about 7 kp to about 25 kp. In some embodiments, the tablets were further coated with a coating agent (e.g., a polyvinyl alcohol based non-functional coating) to produce film-coated fixed dose combination tablets.

The tablets were prepared using the approximate amounts of active and inactive components set forth in Table 1.

TABLE 1
Preparation of an Immediate Release Multi-Unit Tablet of Valsartan and
Nebivolol

| Component | % of layer | mg/Tablet |
|---|---|---|
| First layer | | |
| Valsartan USP | 76.0 | 320.0 |
| Microcrystalline Cellulose | 13.9 | 58.6 |
| Croscarmellose Sodium, NF | 4.8 | 20.1 |
| Hydroxypropyl Methylcellulose | 2.4 | 10.2 |
| Polysorbate 80 | 0.4 | 1.5 |
| Colloidal Silicon dioxide, NF | 1.0 | 4.2 |
| Talc, USP | 0.5 | 2.1 |
| Magnesium Stearate - Sodium Lauryl Sulfate | 1.0 | 4.2 |
| Total for First Layer | 100.0 | 420.9 |
| Second layer | | |
| Nebivolol HCl | 4.3 | 10.8 |
| Lactose Monohydrate, NF | 60.9 | 152.2 |
| Silicified Microcrystalline Cellulose | 25.0 | 62.5 |
| Croscarmellose Sodium NF | 5.0 | 12.5 |
| Colloidal Silicon dioxide, NF | 0.5 | 1.20 |
| Hydroxypropyl Methylcellulose | 2.0 | 5.0 |
| Talc NF | 1.0 | 2.5 |
| Magnesium Stearate - Sodium Lauryl Sulfate | 1.0 | 2.5 |
| Dye | 0.3 | 0.8 |
| Total for First Layer | 100.0 | 250.0 |
| TOTAL TABLET WEIGHT | | 670.9 |

### Example 2

### Preparation of a Dosage Form Comprising Valsartan and Nebivolol (Comparative)

Tablets containing valsartan and nebivolol were prepared using fluid bed top spray granulation followed by drying and blending.

Valsartan was mixed with excipients such as starch, lactose monohydrate, croscarmellose sodium, talc and silicon dioxide in a blender, to produce a pre-blend. The pre-blend was then granulated using a fluid bed top spray process, using a granulation solution containing nebivolol hydrochloride, a hypromellose binder (Methocel E15LV) and a Polysorbate 80 wetting agent (Tween 80).

The granules were then dried in a continuous process in the Glatt fluid bed. LOD for the final granules was approx. 2%-3%. The milled granules were then mixed in a blender with croscarmellose sodium, talc, microcrystalline cellulose, Ster-O-Wet® and sodium lauryl sulfate to produce a final blend.

The final blend was compressed using a Korsch PH106 tablet press at pre-determined weight to produce nebivolol-valsartan tablets having different hardness values ranging from 6 kp to 15 kp (depending upon dose strength). The ratio of valsartan to nebivolol in the final tablets was approximately 15:1.

The core tablets were then coated in a Compulab pan coater using a polyvinyl alcohol-based nonfunctional coating, to produce film coated tablets.

### Example 3

### Comparative Dissolution Performance of the Dosage Forms Prepared in Examples 1 and 2

The dissolution performance of the dosage forms prepared in Examples 1 and 2 were assessed in 900 mL of 0.01N HCl solution at a temperature of 37° C. and subjected to agitation using USP Type II apparatus at 50 rpm. In addition, for comparison purposes, the dissolution performance of Bystolic® (nebivolol hydrochloride) monotherapy was assessed in the same solution and conditions.

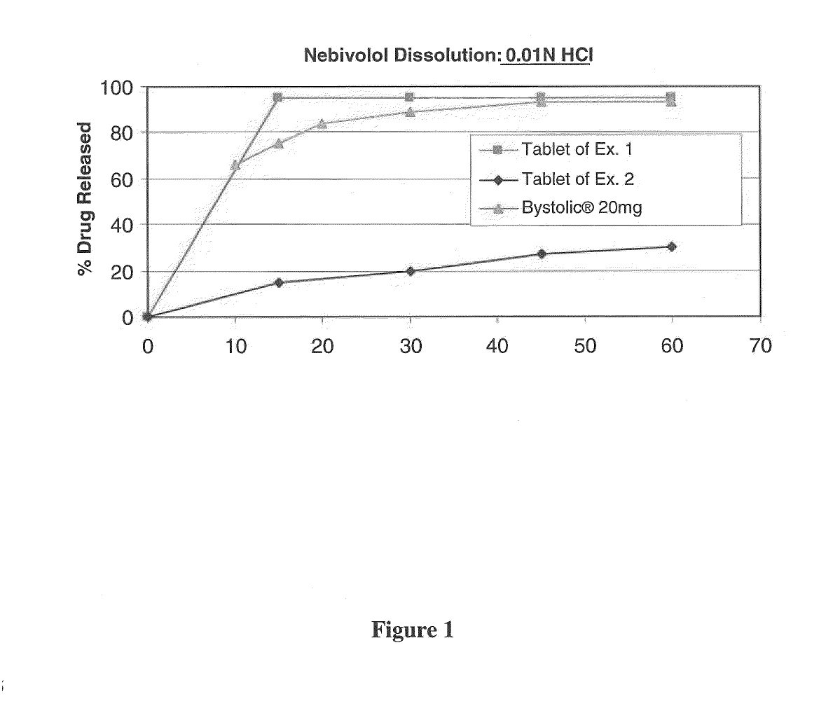

The dissolution rate of nebivolol from the tablets of Examples 1 and 2 are set forth in Table 2 and in FIG. 1.

TABLE 2

% Dissolution of Nebivolol

| | Tablet | Tablet |
|---|---|---|
| Time | prepared in | Prepared in |
| (minutes) | Ex. 1 | Ex. 2 |
| 15 | 95 | 15 |
| 30 | 95 | 20 |
| 45 | 95 | 27 |
| 60 | 95 | 30 |

As is demonstrated in the table, the nebivolol dissolution achieved by the dosage formt of example 1 was unexpectedly greater than that achieved by the dosage form of example 2.

### Example 4

### Stability of the Oral Multi-Unit Dosage Form Prepared in Example 1

The stability of the dosage form prepared in example 1 was assessed initially and following storage of the dosage form without desiccant for two months at 40° C. and 75% relative humidity (RH).

Table 3 shows the approximate concentrations of impurities that were found in the oral dosage forms initially and after two months of storage.

TABLE 3

| | Initial | | | |
|---|---|---|---|---|
| | (t = O months) | | After 2 Months | |
| | Valsartan | Nebivolol | Valsartan | Nebivolol |
| | Layer | Layer | Layer | Layer |
| Assay | 97.4 | 101.4 | 99 | 95.2 |
| (% active) | | | | |
| Total % | 0 | 0.3 | 0 | 0.25 |
| Degradants | | | | |

### Example 5

### Comparative Nebivolol Exposure Achieved in Subjects Receiving Nebivolol (A) Alone and (B) in Free Combination with Valsartan

A single-dose, 3-way crossover trial was performed in healthy subjects to assess the pharmacokinetic (pK) and pharmacodynamic (pD) impact of administered valsartan on nebivolol.

28 subjects aged 19-45 were randomized to the following once-daily treatments for 7 days under fasted conditions: (A) nebivolol alone (20 mg tablet) and (B) concomitant nebivolol 20 mg+valsartan 320 mg in free combination. Blood samples were collected from each subject for pK analysis at the following time points: 0 hour (predose) on Days 1, 14, and 27 and at 0 hour (predose) and 1, 2, 4, 6, 10, 16, 24, 36, and 48 hours postdose on Days 7, 20, and 33.

The bioanalytical procedure used to measure plasma concentrations of nebivolol was a LC/MS/MS (liquid chromatography/tandem mass spectrometry) method. The method was validated to demonstrate the accuracy; linearity, reproducibility, and precision of the analytic procedure.

Among the pK parameters measured were AUC_{0-τ,ss} (area under the plasma concentration versus time curve from time 0 to the dosing interval, τ, at steady state); Cd_{max,ss} (maximum plasma drug concentration at steady state); T_{max,ss} (time of maximum plasma drug concentration following drug administration at steady state); C_{min,ss} minimum plasma drug concentration at steady state); C_{av,ss} (average plasma drug concentration at steady state); and CL/F (apparent total clearance from plasma after oral administration). These pK parameters were derived from plasma concentrations using noncompartmental analysis with the software program SAS (version 9.1.3). In addition, the 2-sided 90% confidence interval (CI) for the ratio of geometric means of Cmax and AUC0-τ,ss between nebivolol coadministered with valsartan and nebivolol alone were constructed.

Table 4 sets forth the assessed nebivolol plasma levels after administration alone and in free combination with valsartan.

TABLE 4
Mean (percent coefficient of variation) nebivolol pK parameters following 20-
mg nebivolol once-daily for 7 days administered alone or concomitantly in free
combination with 320 mg valsartan once-daily for 7 days

| | | | Geometric | 90% |
|---|---|---|---|---|
| | Nebivolol Alone | Nebivolol + Valsartan | least squares | Confidence |
| | Arithmetic Mean | Arithmetic Mean | (LS) Mean | Interval or |
| PK Parameter | (% CV) | (% CV) | Ratio (C/A) | p-Value |
| AUC_{0-τ} | 41.50 | 34.24 | 0.83 | (78.6-88.4) |
| (ng · h/mL) | (71.72) | (68.66) | | |
| Cₘₐₓ | 8.02 | 4.50 | 0.55 | (48.6-63.1) |
| (ng/mL) | (43.26) | (43.58) | | |

As is demonstrated in the table, a surprisingly large pK interactive effect occurred between valsartan and nebivolol when coadministered. Surprisingly, the study revealed that when nebivolol was coadministered with valsartan, the mean nebivolol Cmax and AUC_{0-τ} decreased by as much as about 40-50% and about 15-20%, respectively.

The differences in mean nebivolol T_{1/2} and Tmax between the combination and nebivolol monotherapy were less striking.

### Example 6

Next, a modeling and simulation study was performed to estimate the clinical impact of the surprisingly large pK interactive effect on the therapeutic blood pressure-lowering effects of the drug combination in patients with mild to moderate hypertension.

Surprisingly (as is discussed in this example), the study revealed that despite the unexpectedly large decrease in nebivolol Cₘₐₓ that occurs when nebivolol is coadministered with valsartan (see example 5), this large Cmax drop corresponds to a surprisingly and unexpectedly small decrease in the diastolic blood pressure (DBP) treatment effect.

The study involved first determining the relationship between nebivolol plasma concentration and the therapeutic diastolic blood pressure (DBP)-reducing effect by nebivolol when administered alone. A population PK/PD analysis was performed to assess the relationship between nebivolol plasma concentration and its therapeutic DBP-reducing effect. The analysis utilized PK/PD data that was collected from patients with mild to moderate hypertension following a Phase 3, double-blind, placebo-controlled, multicenter study. During the study, patients were randomized to 7 parallel treatment arms wherein the patient received either a placebo or any of 6 different doses of nebivolol (between 1.25 and 40 mg). The patients were also stratified across all treatment arms by covariates of race (African American vs non-African American), age (<65 vs≧65 years), gender, diabetes status (history of diabetes mellitus vs no history of diabetes mellitus), and by metabolism of nebivolol (PM vs EM of CYP 2D6). Blood pressure data was collected from each patient initially and at days 28 and 84 of treatment.

The study results were refined and updated using nonlinear mixed-effects modeling (NONMEM) software, version VII, with first-order conditional estimation/interaction method. An Emax (saturable maximum effect) model was utilized to determine the relationship between d,l-nebivolol concentration and mean sitting DBP in patients. Moreover, the EC50 (half maximal effective concentration) was determined.

Using this DBP PK/PD model, the EC50 estimate of d,l-nebivolol concentrations was determined to be 0.0179 ng/mL. Moreover, the Emax in DBP reduction was estimated to be approximately 5 mm Hg after correction for the placebo effect. No covariate effects were found to have a significant effect on the DBP PK/PD relationships.

Second, simulation studies were performed with the Emax model to assess whether any deleterious impact on nebivolol-related DBP-lowering effect are expected to occur as a result of the PK interactive effect with valsartan. Posterior simulations were conducted over a range of Cₘₐₓ (20%-80%) and C_{avg} (20%-50%) nebivolol exposure reductions for nebivolol doses of 5, 10, and 20 mg. For purposes of the study, it was assumed for the simulation that valsartan does not have an effect on the nebivolol-DBP exposure-response relationship and only has an effect on nebivolol pharmacokinetics.

Based on the principle of superposition, a reduction in C_{avg} would translate into a corresponding decrease in AUC and AUCₛ. PK data from a Phase 1 open-label, multiple-dose study assessing the pharmacokinetics of nebivolol in healthy volunteers was used to estimate the mean values of d,l-nebivolol CSS,avg and CSS,max in absence of interaction. The metric used to assess the impact of the various reductions in CSS,avg and CSS,max for different nebivolol doses was the decrease in DBP effect, which was calculated using the following equation:

$$Decrease in DBP effect ( mmHg ) = \vert \frac{{E}_{max} \cdot {C}_{dose , noDDI}}{{EC}_{50} + {C}_{dose , noDDI}} - \frac{{E}_{max} \cdot {C}_{dose , DDI}}{{EC}_{50} + {C}_{dose , DDI}} \vert$$

in which:
Cdose, noDDI is the CSS,avg or CSS,max for a given dose with no PK drug-drug interaction (DDI)
Cdose, DDI is the reduced CSS,avg or CSS,max for a given dose due to a PK drug-drug interaction (DDI)
Together, the studies exhibited an approximate mean reduction in nebivolol C_{avg} and C_{SS,avg} of 20% in the presence of valsartan with corresponding Cₘₐₓ and C_{SS,max} reduction of approximately 50%.

However, despite the surprisingly high drop in nebivolol Cₘₐₓ, the study also revealed a surprisingly and unexpectedly small decrease in the diastolic blood pressure (DBP) treatment effect (as compared with the DBP treatment effects by the same dosage of nebivolol when administered alone without valsartan).

For example, even with the approximate 50% reduction in nebivolol Cmax for the 5 mg dose, the study revealed a median decrease in the typical nebivolol DBP treatment effect of only about 0.05 mm Hg (95% confidence interval: 0.012, 0.13). Therefore, for example, a 5 mg nebivolol dose that normally (without valsartan) would have reduced diastolic blood pressure (DBP) to 120 mmHg (from an initial high BP value) will surprisingly still reduce BP to 120.05 mmHg (despite the lowered nebivolol Cmax when coadministered with valsartan). Similarly, for example, a 5 mg nebivolol dose that normally (without valsartan) would have reduced DBP to 118 mmHg (from an initial high BP value) will surprisingly still reduce BP to 118.05 mmHg. Similarly, for example, a 5 mg nebivolol dose that normally (without valsartan) would have reduced DBP to 109 mmHg (from an initial high BP value) will surprisingly still reduce BP to 109.05. Similarly, for example, a 5 mg nebivolol dose that normally (without valsartan) would have reduced DBP to 100 mmHg (from an initial high BP value) will surprisingly still reduce BP to 100.05.

Similarly, even with the approximate 50% reduction in nebivolol Cmax for the 10 mg dose, the study revealed a median decrease in the typical nebivolol DBP treatment effect of only about 0.025 mm Hg (95% confidence interval: 0.006, 0.069). Therefore, for example, a 10 mg nebivolol dose that normally (without valsartan) would have reduced diastolic blood pressure (DBP) to 120 mmHg (from an initial high BP value) will surprisingly still reduce BP to 120.025 mmHg (despite the lowered nebivolol Cmax when coadministered with valsartan). Similarly, for example, a 10 mg nebivolol dose that normally (without valsartan) would have reduced DBP to 118 mmHg (from an initial high BP value) will surprisingly still reduce BP to 118.025 mmHg. Similarly, for example, a 10 mg nebivolol dose that normally (without valsartan) would have reduced DBP to 109 mmHg (from an initial high BP value) will surprisingly still reduce BP to 109.025. Similarly, for example, a 10 mg nebivolol dose that normally (without valsartan) would have reduced DBP to 100 mmHg (from an initial high BP value) will surprisingly still reduce BP to 100.025.

Similarly, even with the approximate 50% reduction in nebivolol Cmax for the 20 mg dose, the study revealed a median decrease in the typical nebivolol DBP treatment effect of only about 0.013 mm Hg (95% confidence interval: 0.003, 0.035). Therefore, for example, a 20 mg nebivolol dose that normally (without valsartan) would have reduced diastolic blood pressure (DBP) to 120 mmHg (from an initial high BP value) will surprisingly still reduce BP to 120.013 mmHg (despite the lowered nebivolol Cmax when coadministered with valsartan). Similarly, for example, a 20 mg nebivolol dose that normally (without valsartan) would have reduced DBP to 118 mmHg (from an initial high BP value) will surprisingly still reduce BP to 118.013 mmHg. Similarly, for example, a 20 mg nebivolol dose that normally (without valsartan) would have reduced DBP to 109 mmHg (from an initial high BP value) will surprisingly still reduce BP to 109.013. Similarly, for example, a 20 mg nebivolol dose that normally (without valsartan) would have reduced DBP to 100 mmHg (from an initial high BP value) will surprisingly still reduce BP to 100.013.

Based on these surprising and unexpected results, it was concluded that the reduced steady-state nebivolol exposure resulting from the pK drug interaction with valsartan is not believed to be clinically meaningful.

### Example 7

### Determination of the Nebivolol Plasma Exposure Achieved by (i) the Multi-Unit Dosage Form of Example 1 Versus (ii) a Free Combination of Nebivolol and Valsartan

A clinical study was performed to assess whether the oral multi-unit dosage forms prepared in Example 1 achieve comparable (or even bioequivalent) nebivolol exposure to a free combination of nebivolol and valsartan.

70 healthy male and female subjects, aged 18-45 years, were randomized to 2 treatment groups: (i) one group received a “free combination” treatment (consisting of a single dose of one nebivolol 20-mg tablet and one valsartan 320-mg tablet) on day 1, followed by a 7-day washout period, and then received a “multi-unit table” (consisting of a single multi-unit tablet comprising 20 mg of nebivolol hydrochloride and 320 mg of valsartan prepared as described in example 1) on day 8; (ii) the other group received a “multi-unit tablet” on day 1, following by the 7-day washout period, and then received a “free combination” treatment on day 8.

Blood samples were collected from each subject for pK analysis at the following time points: starting on Days 1 and 8: 0 hour (predose) and 0.5, 1, 2, 4, 6, 8, 12, 16, 24, 36, and 48 hours postdose PK blood samples were drawn at the nominal times specified above, relative to the dosing time. Predose samples must be drawn within 15 minutes of the dosing time. Among the pK parameters analyzed to assess nebivolol exposure in subjects were AUC₀₋ₜ (area under the plasma concentration versus time curve from time 0 to time t); AUC_{0-∞} (area under the plasma concentration versus time curve from time from time 0 to infinity); Cmax (maximum plasma drug concentration); Tmax (time of maximum plasma drug concentration); T½ (terminal elimination half-life); and CL/F (apparent total clearance of drug from plasma after oral administration).

The pK parameters for the different treatment schedules were compared by means of an analysis-of-variance model using SAS version 9.1.3 or newer under the UNIX operating system. A general linear model with sequence, subject within sequence, treatment, and period as factors will be used as the basis for the analysis. Statistical inference will be based on log-transformed values for the Cmax, AUC0-∞, and AUC0-t parameters. The two-sided 90% confidence interval (CI) for the ratio of geometric means of Cmax, AUC0-∞, and AUC0-t between the test (the multi-unit tablet) and the reference (the “free combination”) treatments were constructed. Tmax for the test and reference treatments were compared using the Wilcoxon signed rank test. Bioequivalence was concluded when the corresponding 90% confidence intervals of the ratio of geometric means were within the range of about 80% to 125%.

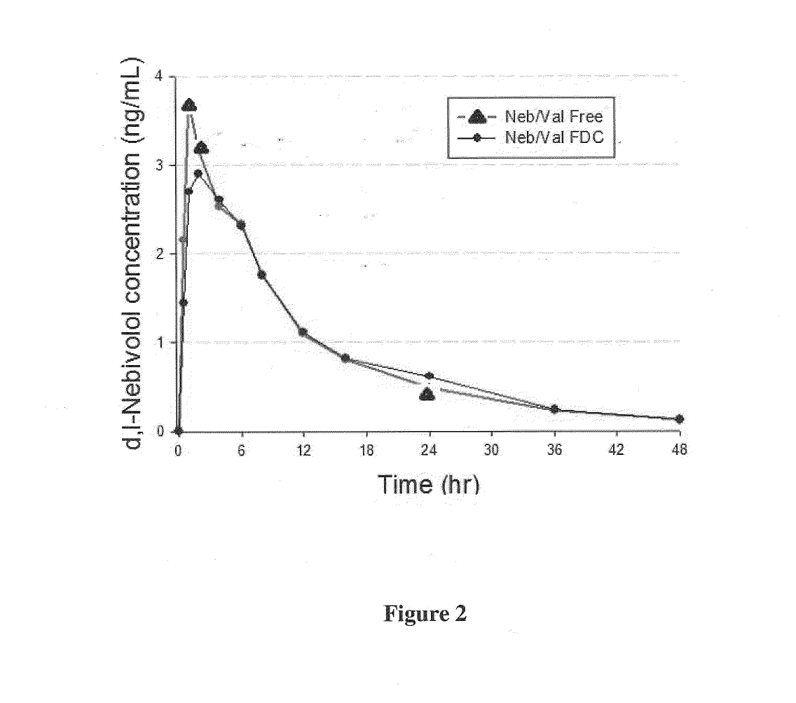

Table 5 and FIG. 2 demonstrate the comparative nebivolol exposure achieved by the free combination versus the multi-unit tablet in terms of the least square mean ratio.

TABLE 5

| | Least Square Mean Ratio |
|---|---|
| | (FDC/Free) |
| | [90% CI] |
| Cmax | 87.3 |
| | [81.7-92.6] |
| AUC₀₋ₜ | 102 |
| | [96.8-107.6] |
| AUC_{0-inf} | 102.3 |
| | [96.6-108.4] |

As is demonstrated in Table 5 and in FIG. 2, the multi-unit tablet was surprisingly discovered to achieve nebivolol Cmax, AUC₀₋ₜ and AUC_{o-inf} 90% confidence intervals that were all within the 80-125% confidence interval range of nebivolol levels from the “free combination” treatment. As was discovered in earlier examples, this high nebivolol exposure achievement by the multi-unit tablet in subjects is related at least in part to the surprising dissolution performance of multi-unit dosage tablet (as is demonstrated with the non-limiting example of a bi-layer tablet in Example 2). Accordingly, the multi-use dosage form discovered to be surprisingly well-suited for achieving therapeutic plasma concentrations of nebivolol in subjects following administration.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure.

The depicted and described embodiments of the invention are exemplary only, and are not exhaustive of the scope of the invention. Consequently, the invention is intended to be limited only by the spirit and scope of the appended claims, giving full cognizance to equivalence in all respects.

What is claimed is:

1. A chemical composition comprising a compound having the structure of formula I: 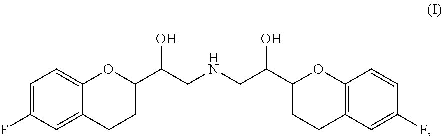 [SMILES: OC(CNCC(O)C1CCc2cc(F)ccc2O1)C1CCc2cc(F)ccc2O1]
a pharmaceutical carrier, and valsartan or a pharmaceutically acceptable salt thereof, wherein the chemical composition upon administering to a patient in need thereof provides a therapeutically effective plasma concentration of the compound of formula I.

2. The chemical composition of claim 1, wherein the composition comprises the component of formula I as a hydrochloride salt.

3. A method of treating hypertension in a patient, comprising administering to a patient diagnosed with hypertension an oral pharmaceutical dosage form that comprises a pharmaceutically acceptable carrier, about 5 mg to about 20 mg of nebivolol or a pharmaceutically acceptable salt thereof, and about 80 mg to about 320 mg of valsartan or a pharmaceutically acceptable salt thereof,
wherein the oral pharmaceutical dosage form upon administering to the patient provides a therapeutically effective plasma concentration of nebivolol for treating hypertension in the patient.

4. The method of claim 3, wherein the oral pharmaceutical dosage form provides a nebivolol Cmax of about 0.5 to about 6 ng/mL following a single administration of the oral pharmaceutical dosage form to a patient in need thereof.

5. The method of claim 4, wherein the oral pharmaceutical dosage form provides:
a nebivolol AUC_{0-∞} for nebivolol of about 5 to about 70 ng·h/mL, and
a nebivolol Tmax of 1 to about 5 hours,
following a single administration of the oral pharmaceutical dosage form to a patient in need thereof.

6. The method of claim 3, wherein the oral pharmaceutical dosage form provides a nebivolol Cmax of about 0.75 to about 5 ng/mL following a single administration of the oral pharmaceutical dosage form to a patient in need thereof.

7. The method of claim 6, wherein the oral pharmaceutical dosage form provides
an AUC_{0-∞} for nebivolol of about 7 to about 60 ng·h/mL, and
a Tmax of 2 to about 4 hours,
following a single administration of the oral pharmaceutical dosage form to a patient in need thereof.

8. The method of claim 3, wherein the oral pharmaceutical dosage form comprises nebivolol or a pharmaceutically acceptable salt thereof in an amount of about 20 mg, and wherein the oral pharmaceutical dosage form provides a nebivolol Cmax between about 3 ng/mL and about 5 ng/mL following a single administration of the oral pharmaceutical dosage form to a patient in need thereof.

9. The method of claim 8, wherein the oral pharmaceutical dosage form provides
an AUC_{0-∞} for nebivolol between about 35 ng·h/mL and about 60 ng·h/mL, and
a Tmax for nebivolol between about 2 hours and about 4 hours,
following oral administration of a single dose of the oral dosage form to a patient in need thereof.

10. The method of claim 8, wherein the oral pharmaceutical dosage form comprises valsartan in an amount of about 320 mg.

11. The method of claim 9, wherein the oral pharmaceutical dosage form comprises valsartan in an amount of about 320 mg.

12. The method of claim 3, wherein the oral pharmaceutical dosage form comprises nebivolol or a pharmaceutically acceptable salt thereof in an amount of about 10 mg, and wherein the oral pharmaceutical dosage form provides a nebivolol Cmax between about 1.5 ng/mL and about 2.5 ng/mL following oral administration of a single dose of the oral dosage form to a patient in need thereof.

13. The method of claim 12, wherein the oral pharmaceutical dosage form provides
an AUC_{0-∞} for nebivolol between about 15 ng·h/mL and about 30 ng·h/mL, and
a Tmax for nebivolol between about 2 hours and about 4 hours,
following oral administration of a single dose of the oral dosage form to a patient in need thereof.

14. The method of claim 12, wherein the oral pharmaceutical dosage form comprises valsartan in an amount of about 160 mg or about 320 mg.

15. The method of claim 13, wherein the oral pharmaceutical dosage form comprises valsartan in an amount of about 160 mg or about 320 mg.

16. The method of claim 3, wherein the oral pharmaceutical dosage form comprises nebivolol or a pharmaceutically acceptable salt thereof in an amount of about 5 mg, and wherein the oral pharmaceutical dosage form provides a nebivolol Cmax between about 0.75 ng/mL and about 1.25 ng/mL following oral administration of a single dose of the oral dosage form to a patient in need thereof.

17. The method of claim 16, wherein the oral pharmaceutical dosage form provides
an AUC_{0-∞} for nebivolol between about 7 ng·h/mL and about 15 ng·h/mL, and
a Tmax for nebivolol between about 2 hours and about 4 hours,
following oral administration of a single dose of the oral dosage form to a patient in need thereof.

18. The method of claim 16, wherein the oral pharmaceutical dosage form comprises valsartan in an amount of about 80 mg or about 160 mg.

19. The method of claim 16, wherein the oral pharmaceutical dosage form comprises valsartan in an amount of about 80 mg or about 160 mg.

20. The method of claim 8, wherein the oral pharmaceutical dosage form releases at least 80% of the nebivolol contained therein after 30 minutes of entering a use environment.

21. The method of claim 12, wherein the oral pharmaceutical dosage form releases at least 80% of the nebivolol contained therein after 30 minutes of entering a use environment.

22. The method of claim 16, wherein the oral pharmaceutical dosage form releases at least 80% of the nebivolol contained therein after 30 minutes of entering a use environment.
